(19) United States
(12) Patent Application Publication
(10) Pub. No.: US 2025/0387252 A1
(43) Pub. Date: Dec. 25, 2025

(54) Orthotic Leg Brace

(71) Applicant: Limbology LLC, Eagle, ID (US)

(72) Inventors: Richard Sydney Miltenberger, Eagle, ID (US); Matthew Shomper, Xenia, OH (US); Steven Mitchell Szymeczek, Boise, ID (US)

(73) Assignee: Limbology LLC, Eagle, ID (US)

(21) Appl. No.: 19/244,439

(22) Filed: Jun. 20, 2025

Related U.S. Application Data
(60) Provisional application No. 63/663,131, filed on Jun. 22, 2024.

(51) Int. Cl. A61F 5/01

(52) U.S. Cl. CPC A61F 5/0111

(57) ABSTRACT

Provided herein are a load-adaptive strut for an orthotic or prosthetic device, a device for supporting or replacing a patient's limb, and a method of designing such devices. The strut comprises a structural element configured by computational design using finite element analysis to adjust load transfer and energy return profiles based on simulation data derived from patient-specific parameters, and adjustable connection points allowing variations in compound angles, offsets, and/or depths. Also provided are embodiments in which means for adjusting load transfer and energy return profiles and means for adjusting connection points are disclosed. The method comprises obtaining patient-specific parameters, performing finite element analysis to determine a computationally designed geometry that modulates load transfer and energy return profiles, and configuring adjustable connection points with compound angles, offsets, or depths based on simulation data.

This application claims the benefit of priority of the U.S. Provisional Patent Application Ser. No. 63/663,131 filed on Jun. 22, 2024, the disclosure of which is incorporated by reference in its entirety for all purposes.

The present disclosure relates to the design and development of devices, such as orthoses and prostheses, particularly aimed at enhancing geometry, capacity, load distribution, and flexibility through computational design.

Existing orthotic devices have insufficient adaptability to various physiological environments. Traditional orthotics, such as foot orthoses and ankle-foot orthoses, exhibit limited mechanical compatibility and functional versatility. For instance, conventional ankle-foot orthoses do not adequately redistribute ground reaction forces to alleviate pain in conditions like plantar fasciitis. Many ankle-foot orthoses do not stabilize weak dorsiflexors and plantar flexors. Conventional prosthetic sockets and exoskeletal orthoses rely on rigid components that do not readily adapt to the variable biomechanics of individual patients, or the dynamic stresses imposed during different gait phases. In many conventional devices, load transfer is predetermined during manufacture. Connection geometries remain static despite variations in patient-specific parameters. Similarly, conventional energy storage and release mechanisms, as seen in existing energy return systems, often lead to inefficient propulsion and increased fatigue due to an inability to adequately mimic natural limb function.

The present disclosure addresses these limitations through computational design. The disclosed devices and methods configure components, enhancing their performance. The devices described herein exhibit improved mechanical compatibility and structural robustness, leading to better functionality and patient outcomes. With biocompatible and lightweight materials, such as carbon fibers and advanced polymers, the disclosed devices ensure strength and longevity.

In particular, the present disclosure provides a load-adaptive strut for an orthotic or prosthetic device that comprises a structural element configured by computational design using finite element analysis to adjust load transfer and energy return profiles based on simulation data derived from patient-specific parameters, and adjustable connection points allowing variations in compound angles, offsets, and/or depths.

The present disclosure also provides a load-adaptive strut for an orthotic or prosthetic device that comprises a means for adjusting load transfer and energy return profiles based on simulation data derived from patient-specific parameters, and a means for adjusting connection points, as disclosed herein.

The present disclosure further provides a device for supporting or replacing a patient's limb that comprises, as disclosed herein, the load-adaptive strut and a structural assembly configured to modulate load distribution and energy return for the patient's limb.

The present disclosure provides a method of designing a strut or device for supporting or replacing a patient's limb that comprises obtaining patient-specific parameters that include at least one value chosen from height, weight, activity level, center of mass, or gait parameters; performing finite element analysis to determine a computationally designed geometry that modulates load transfer and energy return profiles; and configuring adjustable connection points with compound angles, offsets, or depths based on simulation data, as disclosed herein.

Additional embodiments and features are set forth in part in the description that follows, and in part will become apparent to those skilled in the art upon examination of the specification or may be learned by the practice of the embodiments discussed herein. A further understanding of the nature and advantages of certain embodiments may be realized by reference to the remaining portions of the specification and the drawings, which form a part of this disclosure.

### BRIEF DESCRIPTION OF THE DRAWINGS

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

FIG. 1 shows the geometry input for the computational design of an orthotic leg brace disclosed herein.

FIG. 2 shows the variable input for the computational design of the orthotic leg brace of FIG. 1. The variable input includes top point, bottom point, strike point, top box adjustment, bottom box adjustment, anatomy offset, step force, initial point count, start lattice thickness, shell thickness, and radius.

FIG. 3 shows the initial strut generation for the orthotic leg brace of FIG. 1.

FIG. 4 shows the solid-state assessment for the orthotic leg brace of FIG. 1.

FIG. 5 shows the solid optimization for the orthotic leg brace of FIG. 1.

FIG. 6 shows the lattice fill for the orthotic leg brace of FIG. 1.

FIG. 7 shows the strut analysis for the orthotic leg brace of FIG. 1.

FIG. 8 shows the functional grade for the orthotic leg brace of FIG. 1.

FIG. 9 shows the finalization and verification for the orthotic leg brace of FIG. 1.

FIG. 10 shows a cross-section of the orthotic leg brace designed in FIGS. 1-9.

FIG. 11 shows an isometric view of a space-filling model of the orthotic leg brace designed in FIGS. 1-9.

FIG. 12 shows a front view of a space-filling model of the orthotic leg brace designed in FIGS. 1-9.

FIG. 13 shows a side view of a space-filling model of the orthotic leg brace designed in FIGS. 1-9.

FIG. 14 shows the front view of a wireframe model of the orthotic leg brace designed in FIGS. 1-9 (corresponding to the same perspective as FIG. 12).

FIG. 15 shows the side view of a wireframe model of the orthotic leg brace designed in FIGS. 1-9 (corresponding to the same perspective as FIG. 13).

FIG. 16 shows a photograph of the orthotic leg brace depicted in FIGS. 11-15 (corresponding to the same perspective as FIG. 11).

FIG. 17 is a flowchart showing the workflow for orthotic and prosthetic device design disclosed herein.

TABLE 1
Reference Numerals

| | |
|---|---|
| 100 | orthotic leg brace |
| 101 | foundation geometry input |
| 102 | shell geometry input |
| 110 | foundation |
| 120 | (calf) shell |
| 200 | strut |
| 201 | initial strut |
| 202 | solid assessment strut |
| 203 | solid optimization strut |
| 204 | lattice fill strut |
| 205 | functional graded strut |
| 206 | analyzed strut |
| 211 | distal end |
| 212 | first foundation-strut fastener |
| 214 | second foundation-strut fastener |
| 215 | foundation-strut spacing |
| 221 | proximal end |
| 222 | first shell-strut fastener |
| 224 | second shell-strut fastener |
| 225 | shell-strut spacing |
| 300 | variable input |
| 301 | top point |
| 302 | bottom point |
| 303 | strike point |
| 304 | top box adjustment |
| 305 | bottom box adjustment |
| 306 | anatomy offset |
| 307 | step force |
| 308 | initial point count |
| 309 | start lattice thickness |
| 310 | shell thickness |
| 311 | radius |
| 312 | strut diameter |
| 313 | strut material |
| 314 | convergence factor |
| 400 | patient's leg |
| 500 | workflow |
| 510 | input patient-specific geometry |
| 520 | set input variables |
| 530 | generate the initial strut |
| | (design space) |
| 532 | first simulation |
| 534 | first iterative loop |
| 536 | first assessment |
| 540 | refine the strut |
| 550 | create a grid for the structure |
| 560 | apply macrostructure (lightweight) |
| 562 | second simulation |
| 564 | second iterative loop |
| 566 | second assessment |
| 570 | functionally grade the structure |
| 580 | run a final FEA check |
| 590 | export geometry |
| 595 | part file |

### DETAILED DESCRIPTION

Existing designs employ a carbon strut between thermoformed or printed shells, yet the materials are either too flexible or too stiff and do not provide sufficient energy return. Unibody designs use prepreg composites, which result in high costs and unpredictable performance. Multi-piece designs suffer from imprecise, unstable connection points. Prosthetists spend hours fitting and finishing each device. Conventional three-dimensional (3D) printed designs do not endure the force levels that carbon fiber braces manage. Current single-piece 3D design is overly flexible, while conformal designs, despite improvements, still lack lasting comfort and durability.

To overcome these limitations, simulation data are integrated into a design method that directly tailors a lightweight, functionally graded structure for patient-specific load transfer. This feature is not only absent in the prior art but also yields improved operational performance of the prosthetic or orthotic device.

Specifically, the present disclosure provides a computational ankle-foot orthosis design that offers tailored properties for patient needs. The design considers factors such as height, weight, activity level, center of mass, and gait parameters. A multi-step process through finite element analysis fine-tunes the geometry for each patient. Connection points-including compound angles, offsets, and depths-are adjustable on the fly, with the physics model automatically updated.

As used herein, “orthotic device” refers to a device that supports or corrects the alignment and function of a patient's limb with mechanical stabilization and load modulation. In certain embodiments, an orthotic device is configured to support, stabilize, or assist a limb during movement and weight-bearing activities. Suitable examples include, but are not limited to, ankle-foot orthoses, knee orthoses, orthoses that support the entire lower extremity such as unibody devices, foot orthoses, braces for the upper limb, and other devices that provide mechanical support and enhance function during use.

As used herein, “prosthetic device” refers to a device designed to replace a missing or non-functional body part to restore function and/or appearance. In certain embodiments, a prosthetic is configured to replicate the biomechanical properties and/or operational dynamics of the absent part. Suitable examples include, but are not limited to, prosthetic limbs, artificial heart valves, hip replacements, ocular prostheses, facial prostheses, dental prostheses, and cochlear implants. In certain embodiments, the prosthetic device is a prosthetic limb or a portion thereof, configured to mimic natural limb motion, provide stability support, and/or improve mobility during weight-bearing activities. Suitable examples include, but are not limited to, transtibial prostheses, transfemoral prostheses, upper limb prostheses, lower limb prostheses that integrate an artificial socket, strut and joint structure, partial limb prostheses, and customized prosthetic devices designed based on patient-specific measurements.

Without wishing to be bound by theory, the computational and generative design of the geometry for the device incorporates inputs from gait analysis, center of mass, height, weight, activity level, etc. of the patient. In these analyses, the focus is on the energy return of the device, as well as a flexibility/strength ratio that is personalized, being calculated for the gait of the patient receiving the device.

As used herein, “structural element” refers to a component of an orthotic or prosthetic device that provides mechanical support and defines the geometry for load transfer and energy return. In certain embodiments, a structural element achieves a specific physical framework that modulates mechanical forces during use. Suitable examples include, but are not limited to, a functionally graded lattice structure, a unibody framework, a cross-sectional beam, or a load-bearing surface integrated into the overall device.

As used herein, “computational design” refers to the process of developing device geometries and structural configurations using digital tools and analysis methods. In certain embodiments, computational design is implemented with computer algorithms and simulation software to determine load transfer and energy return profiles based on patient-specific data. Suitable examples include, but are not limited to, computer-aided design, finite element analysis, and other simulation-based approaches to producing digital models.

As used herein, “finite element analysis” or “FEA” refers to a computer-aided simulation method that divides a device's structural framework into discrete elements to analyze mechanical stresses and performance under simulated loading conditions. In certain embodiments, finite element analysis is implemented using dedicated software to determine how a structural element responds to applied forces and/or to assess load transfer and energy return profiles based on simulation data.

As used herein, “FEA check” refers to a process that validates and refines finite element analysis models to ensure they reliably predict mechanical behavior under real-world conditions. In certain embodiments, an FEA check compares simulation outputs such as stress distribution, deformation, and force-displacement curves, against experimental data obtained through bench-top mechanical testing following ASTM standards, such as ASTM D790. The FEA check evaluates realistic boundary conditions, including residual limb-socket friction and dynamic gait-phase loads, and verifies material behavior by calibrating stress-strain data for simulating hyperelastic or viscoelastic responses. In addition, mesh sensitivity is assessed during an FEA check by employing appropriate hexahedral or tetrahedral elements to ensure convergence and accuracy for complex geometries. In various embodiments, simulation benchmarks reveal stress hotspots, displacement, energy return ratios, and structural safety metrics correlated with clinical outcomes, enabling iterative design and reducing the need for trial-and-error prototyping.

As used herein, “load transfer” refers to the process by which forces are distributed through a device from one region to another during weight-bearing or dynamic activities. In certain embodiments, load transfer is defined as the method by which mechanical stresses and loads are directed from a patient's limb to the supporting structure of an orthotic or prosthetic device. Suitable examples include, but are not limited to, the transfer of weight from a residual limb through a prosthetic socket to the attached strut and supporting assembly, and the distribution of ground reaction forces across an ankle-foot orthosis.

As used herein, “energy return profile” refers to the characteristic of an orthotic or prosthetic device that describes how mechanical energy is stored during the stance phase and released during push-off to aid in propulsion and improve gait. In certain embodiments, the energy return profile is determined by the device's ability to capture energy during loading and to return a portion of that energy during unloading to reduce the work performed by the user. Suitable examples include, but are not limited to, energy storage and return feet made, spring-like ankle-foot orthoses that provide measurable energy return during push-off, and other designs that offer a quantifiable mechanism for energy storage and release.

As used herein, “simulation data” refer to data generated from computer-based models that represent device behavior under various load and dynamic conditions. Suitable examples include, but are not limited to, numerical stress distributions, deformation profiles, and dynamic force outputs generated from simulated gait cycles.

As used herein, “connection geometry” refers to the spatial arrangement and configurational features of connection points and interfaces that join components within an orthotic or prosthetic device. In certain embodiments, connection geometry is defined by the dimensions, orientation, and relative positions of adjustable connection points that allow for modifications of alignment and load transfer across the device. Suitable examples include, but are not limited to, angled joints, sliding interfaces, repositionable fixtures, and adjustable fittings that establish how a strut, structural assembly, and/or socket interconnects with other elements.

As used herein, “compound angle” refers to an angle formed by the intersection of two or more planes that result in a multi-dimensional relationship between components in an orthotic or prosthetic device. In certain embodiments, a compound angle is defined by the combined angular orientations adjustable through connection points to align structural elements. Suitable examples include, but are not limited to, configurations where a strut is connected at an angle that includes both a medial-lateral and an anterior-posterior component, and arrangements where a joint connection permits simultaneous pitch and yaw adjustments.

As used herein, “offset” refers to the displacement between corresponding components or connection points, thereby shifting from a predetermined reference alignment in an orthotic or prosthetic device. In certain embodiments, an offset is defined as the physical distance by which one element is positioned laterally or longitudinally relative to an adjacent element or a defined reference axis. Suitable examples include, but are not limited to, a strut connection where the interface is deliberately positioned away from a central axis and mounting features that provide a specific gap between interconnected parts, such as a between a foundation and strut or between a calf shell and a strut.

In certain embodiments, the strut as disclosed herein comprises a structural element that comprises a functionally graded lattice structure exhibiting controlled displacement tailored to patient-specific parameters.

As used herein, “functionally graded lattice structure” or “FGLS” refers to advanced internal architectures that vary in geometry, density, and material properties across different regions of a device to achieve a gradual transition in mechanical characteristics, such as stiffness and strength, which distributes mechanical loads more naturally and minimizes stress concentrations. In certain embodiments, FGLSs are produced by additive manufacturing methods that allow patient-specific customization and fine-tuning of energy absorption and energy return properties to match the biomechanics of the patient. Suitable examples include, but are not limited to, load-adaptive struts, lattice structures integrated within a prosthetic socket that provide a smooth change from high-compression to low-compression regions, and lattice elements in prosthetic feet or ankle-foot orthoses that accommodate varying gait phases by locally adjusting density and porosity for improved shock absorption and/or energy recovery.

As used herein, “patient-specific parameter” refers to a measurement or datum that reflects a characteristic of a patient for tailoring an orthotic or prosthetic device. In certain embodiments, patient-specific parameters are data such as a patient's height, weight, center of mass, gait parameters, limb geometry, residual limb dimensions, and activity level. Suitable examples include, but are not limited to, the measured length and circumference of a residual limb, the angle of limb alignment, the patient's overall stature, and quantified levels of physical activity.

In certain embodiments, the strut as disclosed herein comprises adjustable connection points that are configured for real-time automatic adjustment to update associated physics parameters.

As used herein, “adjustable connection point” refers to a point or interface on an orthotic or prosthetic device configured for geometric modification. In some embodiments, the configuration is performed in real-time. In certain embodiments, an adjustable connection point is designed to accommodate variations in compound angles offsets, and depths to tailor the device's alignment and load transfer characteristics. Suitable examples include, but are not limited to, connection features integrated into a strut or structural assembly that allow mechanical adjustment through sliding joints or repositionable fixtures.

As used herein, “controlled displacement” refers to the deliberately managing movement or deformation within an orthotic or prosthetic device to regulate the timing and magnitude of energy storage and release under load. In certain embodiments, controlled displacement is accomplished using mechanical means, such as springs, or through electronic means, such as actuators, or advanced lattice structures that provide a predetermined response during a user's gait cycle.

As used herein, “real-time automatic adjustment” refers to the immediate and continuous modification of device settings in response to dynamic conditions during use. In certain embodiments, real-time automatic adjustment is provided by control systems or mechanical devices that update parameters, such as connection geometry and load distribution, automatically as a device is operated. Suitable examples include, but are not limited to, systems that adjust connection points based on sensor feedback during a gait cycle, and mechanisms that alter stiffness in response to varying load levels.

As used herein, “configuration module” refers to a component that aids the adjustment of patient-specific design parameters for an orthotic or prosthetic device. In certain embodiments, the configuration module is a hardware device or software tool that receives input regarding factors such as limb dimensions, weight, and activity level and accordingly adjusts connection geometry, load distribution, and energy return profiles. Suitable examples include, but are not limited to, a computer-based interface integrated with design software, a user-operated adjustment mechanism, and an electronic calibration module that automatically sets device parameters.

As used herein, “associated physics parameter” refers to a measured or computed physical variable that governs the mechanical behavior of device components, such as stiffness, elastic modulus, damping, energy return ratio, displacement, and relative density. In certain embodiments associated physics parameters are determined through quantitative analysis to guide the configuration for load transfer and controlled displacement during dynamic operation.

As used herein, “stiffness” refers to the resistance of a material or structure to deformation under an applied force. In certain embodiments, stiffness is expressed in units such as newton-meters per degree or newtons per meter and is used to quantify how much a device flexes when loaded.

As used herein, “elastic modulus” refers to the ratio of stress to strain in a material within its elastic region. In certain embodiments, the elastic modulus is expressed in pascals and quantifies how much a material will stretch or compress under an applied force.

As used herein, “damping” refers to the capacity of a material or structure to dissipate energy during cyclic loading and unloading. In certain embodiments, damping is measured as the rate at which vibrations or oscillations are reduced in response to impact forces.

As used herein, “energy return ratio” refers to the proportion of mechanical energy stored during deformation that is returned during unloading. In certain embodiments, the energy return ratio is quantified as a percentage that indicates how efficiently a device recovers energy during movement.

As used herein, “displacement” refers to the movement or deformation of a component in response to an applied force. In certain embodiments, displacement is measured in millimeters or degrees and indicates how much a structural element moves under load.

As used herein, “relative density” refers to the ratio of the density of a lattice structure to the density of the corresponding solid material. In certain embodiments, relative density is used to tune mechanical properties by indicating the proportion of void space to material within a functionally graded lattice structure.

In certain embodiments, the strut as disclosed herein comprises a structural element formed from one or more materials chosen from carbon fiber, advanced polymers, or composites thereof. In certain embodiments, the device comprises carbon struts.

As used herein, “carbon fiber” refers to a reinforcement material essentially consisting of carbon atoms arranged in long thin fibers. Carbon fiber enhances structural strength and stiffness in orthotic and prosthetic components. In certain embodiments, carbon fiber is provided in continuous form or as chopped reinforcements. In certain embodiments, carbon fiber is combined with advanced polymers to form lightweight high-strength composite structures. Suitable examples include, but are not limited to, T300 carbon fiber, T700 carbon fiber, and high-modulus carbon fibers.

As used herein, “advanced polymer” refers to a high-performance polymer material that exhibits high strength durability and enhanced resistance to impact. In certain embodiments, advanced polymer is a thermoplastic or thermoset material with dimensional stability under stress and the ability to form composite structures with reinforcing fibers. Suitable examples include, but are not limited to, polyetheretherketone (PEEK), polyetherimide (PEI), reinforced epoxy resin, polycarbonate, polyurethane, and polyamide, such as Nylon 11, Nylon 11 CF, and Nylon 12.

As used herein, “polyamide” or “nylon” refers to high-performance thermoplastics used in additive manufacturing for orthotics and prosthetics due to their robust mechanical properties, biocompatibility, and process compatibility. These polymers comprise repeating amide linkages (—CONH—) formed by condensation reactions between amine and carboxylic acid groups. This structure enables strong hydrogen bonding between chains and governs the balance between crystalline regions, which provide strength and thermal resistance, and amorphous regions, which offer elasticity and impact resistance. Variants such as aliphatic (Nylon 6,6 and Nylon 6), aromatic (Kevlar), and semi-aromatic (PPA) types exist, each exhibiting unique balances of strength, flexibility, and thermal properties. In additive manufacturing, these materials deliver high tensile strength, impact resistance, and fatigue endurance; they absorb minimal moisture; and they resist chemicals while sustaining thermal stability. Such features allow the production of intricate lattice structures that are lightweight yet durable.

“Nylon 11,” “Polyamide 11,” or “PA 11” refers to a bioplastic produced by polymerizing 11-aminoundecanoic acid obtained from castor oil. The material offers high strength, toughness, and excellent resistance to impact, chemicals, fuels, and oils. It maintains its performance at elevated temperatures while absorbing minimal moisture for improved dimensional stability. Additionally, Nylon 11 remains ductile and tough at very low temperatures (down to −40° C. or lower).

“Nylon 11 CF” refers to carbon fiber-reinforced nylon powder comprising Nylon 11 As the base polymer and chopped carbon fibers dispersed within the nylon matrix to enhance stiffness and strength. Its mechanical properties are about 69 MPa tensile strength and 5300 MPa (5.3 GPa) tensile modulus, with an elongation at break of about 9%. Additional flexural properties include a flexural strength of about 110 MPa, a flexural modulus near 4200 MPa (4.2 GPa), and a notched Izod impact resistance of about 74 J/m. Thermal performance is characterized by a heat deflection temperature of about 178° C. at an applied load of 1.8 MPa, about 188° C. at an applied load of 0.45 MPa, and by a Vicat softening temperature near 188° C.

“Nylon 12” (also known as “Polyamide 12” or “PA 12”) is a high-performance semi-crystalline thermoplastic known for its mechanical strength, flexibility, chemical resistance, and low moisture absorption. It is synthesized from ω-aminolauric acid or laurolactam monomers containing 12 carbon atoms and is produced primarily via ring-opening polymerization to achieve greater stability. Nylon 12 exhibits minimal water absorption (0.2-0.25%), excellent dimensional stability, strong resistance to oils, fuels, hydraulic fluids, solvents, and saltwater, as well as robust mechanical performance with tensile strengths of up to 50 MPa. Moreover, it provides high impact resistance, fatigue resistance, superior performance over a wide temperature range, low friction, good wear properties, and effective noise and vibration damping.

In certain embodiments, a load-adaptive strut for an orthotic or prosthetic device comprises a means for adjusting load transfer and energy return profiles based on simulation data derived from patient-specific parameters, and a means for adjusting connection points.

In certain embodiments, the strut as disclosed herein comprises the means for adjusting load transfer and energy return profiles that comprise a lattice structure exhibiting controlled displacement tailored to patient-specific parameters.

In certain embodiments, the strut as disclosed herein comprises the means for adjusting connection points that are operable to automatically update associated physics parameters in real-time.

In certain embodiments, the strut as disclosed herein comprises means for adjusting load transfer and energy return profiles that comprise one or more materials chosen from carbon fiber, advanced polymers, and composites thereof.

In certain embodiments, a device for supporting or replacing a patient's limb comprises the load-adaptive strut as disclosed herein, and a structural assembly configured to modulate load distribution and energy return for the patient's limb. In certain embodiments, the device as disclosed herein comprises a structural assembly that comprises an upper section and a lower section operatively coupled to the strut. In certain embodiments, the generative geometry is applied to the upper and lower pieces, not just the strut component.

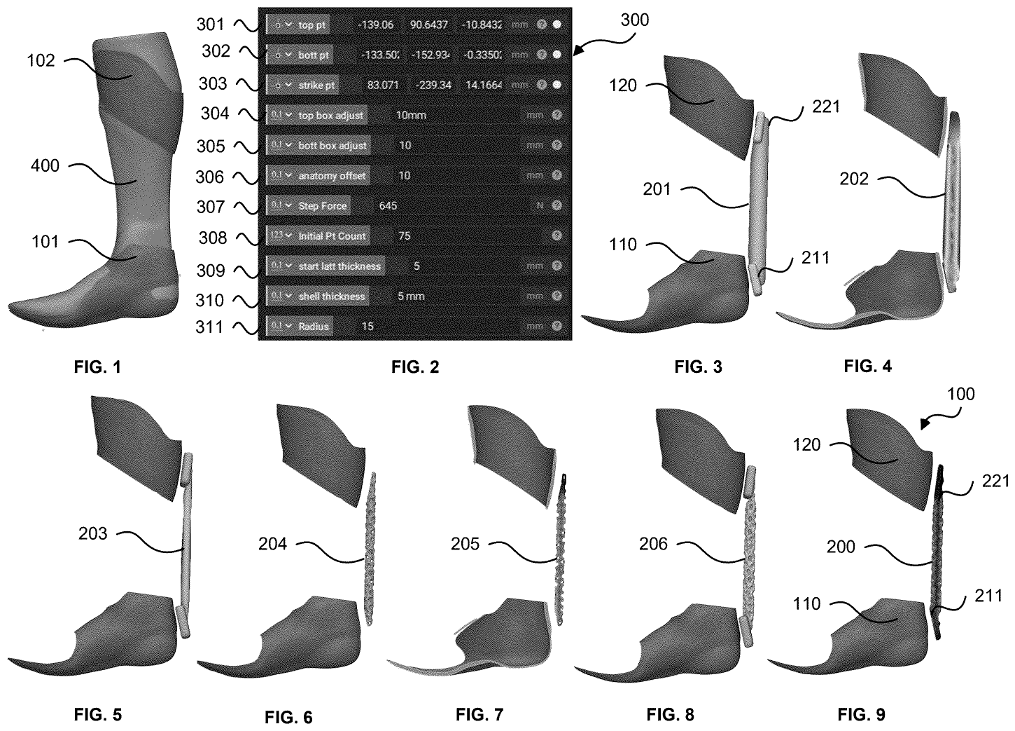

FIG. 1 shows the geometry input for the computational design of an orthotic leg brace 100 disclosed herein. The foundation geometry input 101 and shell geometry input 102 are from a patient's leg 400.

FIG. 2 shows the variable input 300 for the computational design of the orthotic leg brace 100 of FIG. 1. The variable input includes top point 301, bottom point 302, strike point 303, top box adjustment 304, bottom box adjustment 305, anatomy offset 306, step force 307, initial point count 308, start lattice thickness 309, shell thickness 310, and radius 311.

Referring now to FIGS. 3-9, strut 200 of the orthotic leg brace 100 is developed through each step of the workflow disclosed herein. (More on that later.) The orthotic leg brace 100 comprises a foundation 110 operatively coupled to the distal end 211 of the strut 200 and a calf shell 120 operatively coupled to the proximal end 221 of the strut 200. An initial strut 201 is formed, which undergoes a solid-state assessment 202, solid optimization 203, lattice fill 204, functional grading 205, strut analysis 206, and finalization and verification for the strut 200.

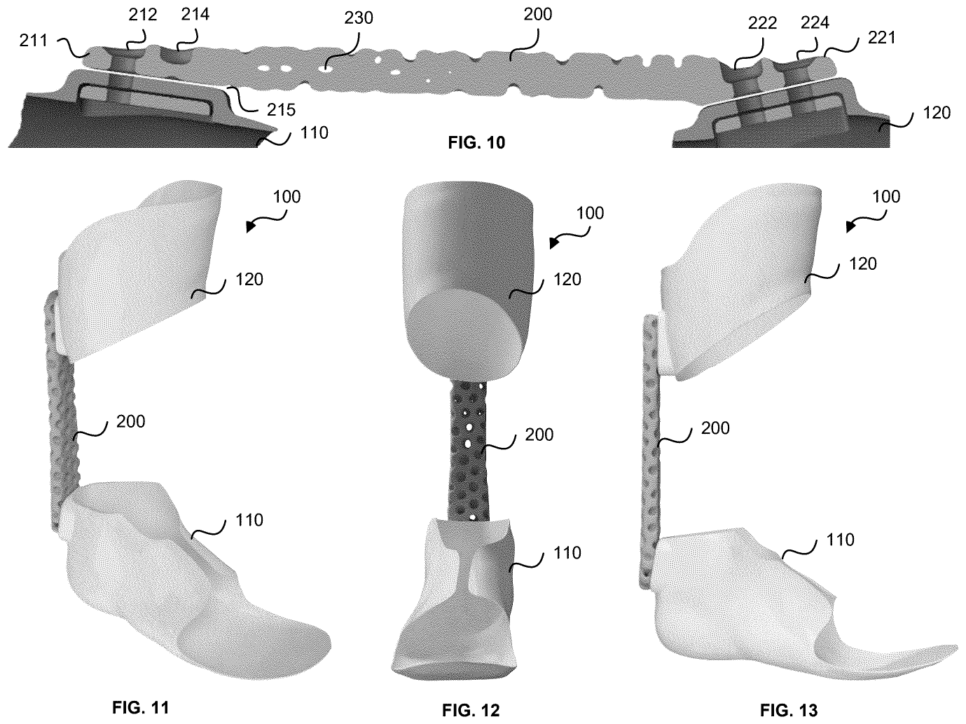

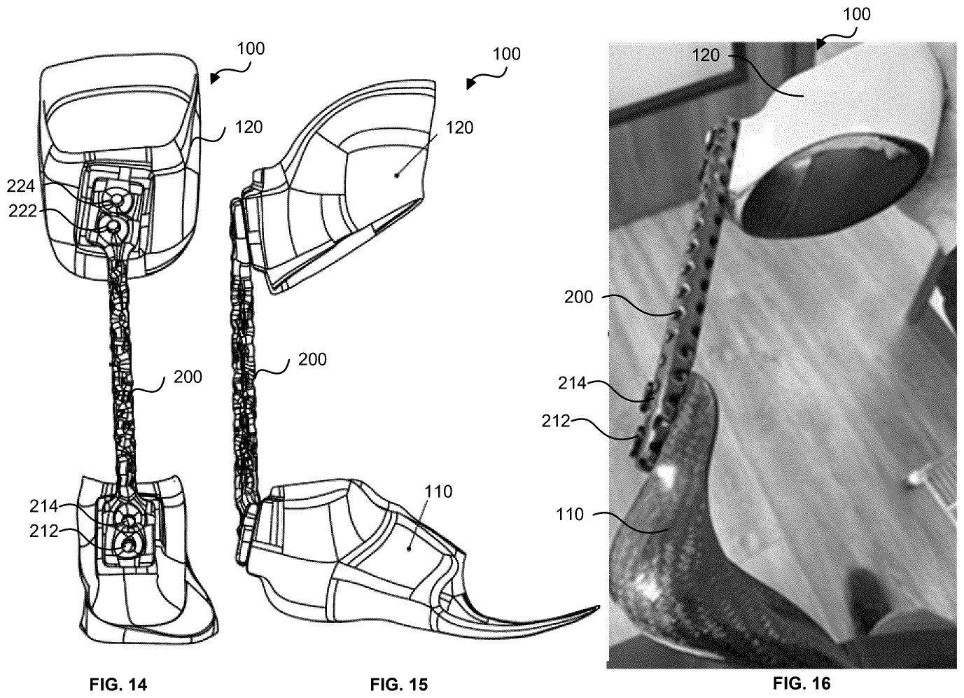

FIG. 10 shows a cross-section of the orthotic leg brace 100 designed in FIGS. 1-9. FIGS. 11-16 show isometric (FIGS. 11 and 16), front (FIGS. 12 and 14), and side perspectives (FIGS. 13 and 15) of the orthotic leg brace 100. The strut 200 comprises an FGLS 230 optimized for energy return. Foundation 110 is operatively coupled to the distal end 211 of the strut 200 via first and second foundation-strut fasteners 212,214, thereby providing algorithmic plate and hole placement and exact spacing 215 between the foundation 110 and the strut 200. Likewise, calf shell 120 is operatively coupled to the proximal end 221 of the strut 200 via first and second shell-strut fasteners 222,224, thereby providing algorithmic plate and hole placement and exact spacing 225 between the shell 120 and the strut 200.

In certain embodiments, the device as disclosed herein comprises a device chosen from an orthotic device, a prosthetic device, an ankle-foot orthosis, a foot orthosis, a knee orthosis, and a knee-ankle-foot orthosis.

In certain embodiments, a hip-knee-ankle-foot orthosis (HKAFO) comprises an orthotic device that spans the hip, knee, ankle, and foot. This orthosis treats patients with pelvic stabilizing trunk muscle weakness, offering support across multiple joints to enhance overall stability and mobility.

In certain embodiments, a relief orthosis comprises a device used to mitigate joint degeneration or support the recovery process after injury or surgery. These orthoses control, guide, limit, or immobilize an extremity, joint, or body segment, aid movement, reduce weight-bearing forces, aid in post-fracture rehabilitation, and correct shape or function to ease movement and reduce pain.

In certain embodiments, an ulcer healing orthosis (UHO) comprises a custom-made ankle/foot orthosis designed to treat foot ulcers. This orthosis features a rigid L-shaped support member with a hollow region to protect the ulcer, and an anterior support shell attached to an articulated hinge, which aids in distributing weight away from the ulcer.

In certain embodiments, a foot orthosis (FO) comprises a device inserted into shoes to provide foot support by redistributing ground reaction forces. These are custom-made and used to alleviate pain in conditions such as high-arched feet, rheumatoid arthritis, plantar fasciitis, first metatarsophalangeal joint pain, or hallux valgus, and to prevent diabetic foot ulcers.

In certain embodiments, an ankle-foot orthosis (AFO) comprises an orthotic device that supports the ankle and foot, often used to immobilize these regions in cases of arthritis or a fracture. AFOs provide stability to weak dorsiflexors and plantarflexors and are the most used orthoses in the United States.

In certain embodiments, a knee orthosis (KO) comprises a device that extends above and below the knee joint to support or align the knee. It stabilizes in cases of diseases causing neurological or muscular impairment, aids in preventing flexion, extension, or instability, and relieves pressure and pain in conditions like arthritis or osteoarthritis.

In certain embodiments, a custom-fabricated product for long-term care comprises custom-made orthoses manufactured or adapted by an orthotist or trained orthopedic technicians per the physician's prescription. This process includes a detailed physical examination and an interdisciplinary discussion to configure orthotic functions and integrate functional elements.

In certain embodiments, a knee-ankle-foot orthosis (KAFO) comprises an orthotic device that spans the knee, ankle, and foot. This orthosis is designed for patients with weakness in knee or hip extensors and includes two orthotic joints-one at the ankle and the other at the knee-to enhance stability.

In certain embodiments, a rehabilitation brace comprises a device used post-injury or following surgery to limit knee movement. These braces often feature an adjustable range of motion to control flexion and extension, and they are larger and designed to immobilize the leg for better recovery.

In certain embodiments, an upper-limb orthosis comprises a mechanical or electromechanical device applied externally to the arm or its segments to restore or improve function or structure. These orthoses address musculoskeletal and neurological impairments affecting the arm due to trauma or disease.

In certain embodiments, a spinal orthosis comprises a device used to treat conditions like scoliosis or spinal fractures. Examples include the Milwaukee brace, Boston brace, Charleston bending brace, Providence brace, and Jewett brace, each designed to provide support and aid in the treatment process.

In certain embodiments, a head orthosis comprises devices such as helmets, used to provide support and protection for the head, commonly in the treatment of specific head injuries or conditions.

In certain embodiments, a robotic transtibial prosthesis comprises a prosthetic limb comprising robotics for mobility through powered ankle-foot motion. Geometry and control systems enable precise actuation, enhancing biomechanics through improved stability, symmetry, and reduced energy expenditure during gait.

In certain embodiments, an energy-storing foot prosthesis comprises a compressible heel unit that stores energy during initial ground contact and releases it during the latter phase to aid in forward propulsion, energy return, and improve walking efficiency and comfort for the patient.

In certain embodiments, a direct bone attachment prosthesis comprises a titanium bolt that is implanted into the residual bone, allowing it to integrate with the bone over time. The computational design optimizes the structural integrity and weight distribution, significantly enhancing muscle control and wear time.

In certain embodiments, a myoelectric prosthesis comprises a limb that converts muscle movements into electrical signals to control the limb's actions. Computational design refines control algorithms, reducing latency and enhancing the naturalness of movement by effectively translating electromyographic signals.

In certain embodiments, a prosthetic limb comprises advanced electronics integrated into the device to provide additional stability and control.

In certain embodiments, a prosthetic socket comprises a socket specifically designed to match the patient's residual limb. Digital scanning captures specific geometry. Load distribution and the fit of the socket increase comfort and reduce wear.

In certain embodiments, a prosthetic limb with active feedback comprises sensors and systems that allow the patient to perceive sensations such as touch or pressure. Computational design integrates sensor networks to provide real-time feedback, thereby enabling natural interaction with the environment.

In certain embodiments, a lightweight prosthetic device comprises advanced materials such as carbon fiber and titanium to reduce weight while ensuring durability. Computational design enhances use of these materials, thereby improving the device's performance and patient comfort.

In certain embodiments, an artificial limb with artificial intelligence (“AI”) integration comprises machine learning algorithms that adapt to the patient's movements over time. The prosthetic continuously adjusts in real-time, offering smoother and more natural motion.

In certain embodiments, the device as disclosed herein further comprises a configuration module for adjusting patient-specific design parameters.

In certain embodiments, the device disclosed herein comprises a unibody orthosis configured as a single printed piece.

As used herein, “unibody orthosis” refers to an orthotic device fabricated as a single integrated unit providing continuous support and stabilization for a patient's limb. In certain embodiments, a unibody orthosis is designed to eliminate component assembly by forming one cohesive structure that delivers consistent mechanical performance and improved weight distribution.

In certain embodiments, the device as disclosed herein comprises a device configured as an ankle-foot orthosis.

In certain embodiments, the device disclosed herein comprises an ankle-foot orthosis that redistributes ground reaction forces to alleviate pain and enhance stability for weakened dorsiflexors and plantarflexors.

As used herein, “ground reaction force” refers to the force exerted by the supporting surface on a device during weight-bearing and movement. In certain embodiments, ground reaction force is measured as the load transmitted through the limb during various phases of gait. It is used to assess and balance force distribution in orthotic and prosthetic devices.

In certain embodiments, a method of designing a strut or device for supporting or replacing a patient's limb comprises obtaining patient-specific parameters that include at least one value chosen from height, weight, activity level, center of mass, or gait parameters; performing finite element analysis to determine a computationally designed geometry that modulates load transfer and energy return profiles; and configuring adjustable connection points with compound angles, offsets, or depths based on simulation data. In certain embodiments, the method as disclosed herein further comprises automatically adapting the adjustable connection points in real-time to update physics parameters. In certain embodiments, the method as disclosed herein comprises applying the computationally designed geometry to configure the device as an orthotic or prosthetic device.

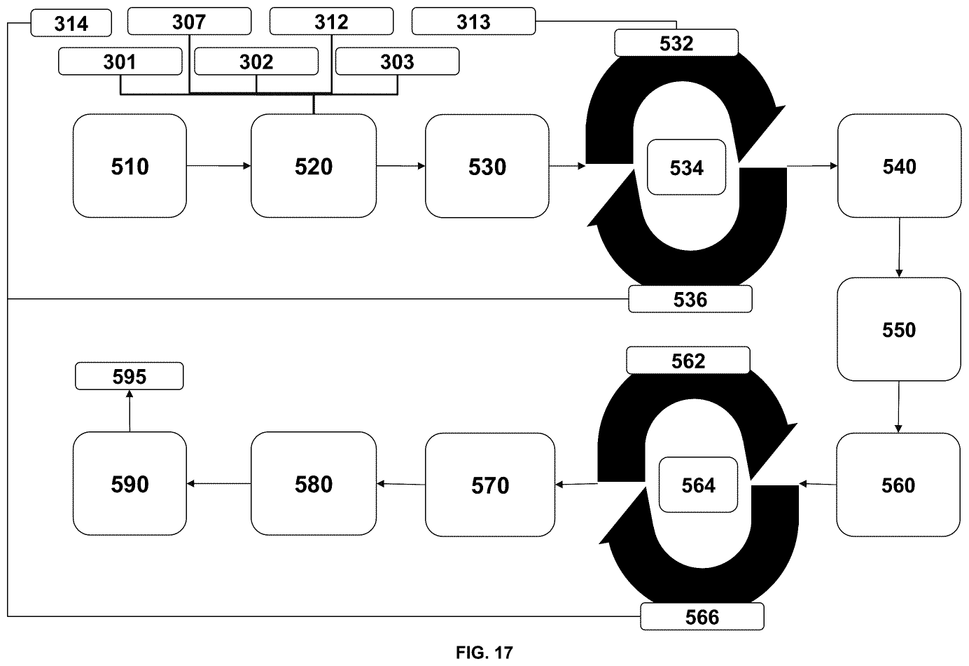

FIG. 17 is a flowchart showing workflow 500 for orthotic and prosthetic device design disclosed herein. The first step is to input patient-specific geometry 510 (such as the foundation and shell geometry inputs 101,102 shown in FIG. 1). The second step is to set input variables 520, for example, top point 301, bottom point 302, strike point 303, step force 307, strut diameter 312, and strut material 313. (Other input variables 300 are shown in FIG. 2.) The third step is to generate the initial strut (design space) 530 (such as the initial strut 201 shown in FIG. 3). A first simulation 532 is run in a first iterative loop 534, which produces a first assessment 536 that generates a convergence factor 314 to feed back into the workflow 500.

Workflow 500 continues with step four to refine the strut 540 (producing, for example, the solid assessment strut 202 of FIG. 4). Step five is to create a grid for the structure 550 (producing, for example, solid optimization strut 203 of FIG. 5). Step six to apply macrostructure (lightweight) 560 (producing, for example, lattice fill strut 204 of FIG. 6). A second simulation 562 is run in a second iterative loop 564, which produces a second assessment 566 that generates a convergence factor 314 to feed back into the workflow 500.

Workflow 500 then proceeds with step seven to functionally grade the structure 570 (producing, for example, functionally graded strut 205 from FIG. 7). Step eight is to run a final FEA check 580 (producing, for example, analyzed strut 206 from FIG. 8). Step nine finalizes and verifies device or device component (such as strut 200 from FIG. 9) to export its geometry 590, producing a part file 595.

In certain embodiments, a device formed by the method as disclosed herein is provided.

Although the disclosure described herein is susceptible to various modifications and alternative iterations, specific embodiments thereof have been described in greater detail above. It should be understood, however, that the detailed description of the composition is not intended to limit the disclosure to the specific embodiments disclosed. Rather, it should be understood that the disclosure is intended to cover all modifications, equivalents, and alternatives falling within the spirit and scope of the disclosure as defined by the claim language.

When introducing elements of the present disclosure or the embodiments(s) thereof, the articles “a,” “an,” “the,” and “said” are intended to mean that there is one or more of the elements. The terms “comprising,” “including,” and “having” are intended to be inclusive and mean that there may be additional elements other than the listed elements.

Having described the disclosure in detail, it will be apparent that modifications and variations are possible without departing from the scope of the disclosure defined in the appended claims.

### EXAMPLES

The following examples are included to demonstrate certain embodiments of the disclosure. It should be appreciated by those of skill in the art that the techniques disclosed in the examples represent techniques discovered by the inventors to function well in the practice of the disclosure. Those of skill in the art should, however, in light of the present disclosure, appreciate that many changes can be made in the specific embodiments that are disclosed and still obtain a like or similar result without departing from the spirit and scope of the disclosure. Therefore, all matter set forth is to be interpreted as illustrative and not in a limiting sense.

### Example 1: Carbon Fiber PDE Struts Stiffness Evaluation

Industry-standard carbon fiber posterior dynamic element (“PDE”) struts rated at #1, #3, and #5 stiffness were evaluated using a crank frame, a calibrated force gauge, and a strut bending fixture configured per ASTM D790 for flexural property measurements. These struts were tested as controls to compare against the load-adaptive struts disclosed herein.

Each PDE strut was mounted in the bending fixture and subjected to a controlled load until reaching a defined maximum safe deflection before material yield. The minimum stiffness in the y-axis was neglected for simplification purposes. As shown in Tables 2-4, imaging at maximum deflection was recorded to document the flexural behavior and verify that the observed deflections matched the expected gradations in stiffness.

TABLE 2
Test results for PDE rated at #1 Stiffness

| Force (lbf) | Position Y (in) | Position X | Δ Deflection Y | Δ Deflection X |
|---|---|---|---|---|
| 0 | 2.875 | 1 | 0 | 0 |
| 10 | 2.75 | 1.5 | −0.125 | 0.5 |
| 20 | 2.625 | 2.125 | −0.125 | 0.625 |
| 30 | 2.625 | 2.875 | 0 | 0.75 |
| 40 | 2.5 | 3.375 | −0.125 | 0.5 |
| Total | | | −0.375 | 2.375 |

The average stiffness in the X direction was 16.8 lbf/inch (1.90 Nm).

TABLE 3
Test results for PDE rated at #3 Stiffness

| Force (lbf) | Position Y (in) | Position X | Δ Deflection Y | Δ Deflection X |
|---|---|---|---|---|
| 0 | 2.9 | 1 | 0 | 0 |
| 10 | 2.875 | 1.25 | −0.025 | 0.25 |
| 20 | 2.75 | 1.5 | −0.125 | 0.25 |
| 30 | 2.75 | 1.875 | 0 | 0.375 |
| 40 | 2.625 | 2.125 | −0.125 | 0.25 |
| 50 | 2.625 | 2.5 | 0 | 0.375 |
| 60 | 2.625 | 2.875 | 0 | 0.375 |
| Total | | | −0.275 | 1.875 |

The average stiffness in the X direction was 32 lbf/inch (3.6 Nm).

TABLE 4
Test results for PDE rated at #5 Stiffness

| Force (lbf) | Position Y (in) | Position X | Δ Deflection Y | Δ Deflection X |
|---|---|---|---|---|
| 0 | 2.875 | 0.875 | 0 | 0 |
| 10 | 2.875 | 1.1 | 0 | 0.225 |
| 20 | 2.75 | 1.375 | −0.125 | 0.275 |
| 30 | 2.75 | 1.5 | 0 | 0.125 |
| 40 | 2.625 | 1.875 | −0.125 | 0.375 |
| 50 | 2.625 | 2 | 0 | 0.125 |
| 60 | 2.625 | 2.2 | 0 | 0.2 |
| 70 | 2.625 | 2.375 | −0.25 | 0.175 |
| Total | | | −0.25 | 1.5 |

The average stiffness in the X direction was 46.7 lbf/inch (5.27 Nm).

### Example 2: Additively Manufactured Nylon 12 Struts Stiffness Evaluation

Two additively manufactured Nylon 12 struts were tested using an apparatus like that used in Example 1, comprising a crank frame, calibrated force gauge, and strut bending fixture conforming to ASTM D790 methods. One strut was oversimplified. The other was produced via the workflow shown in FIG. 17, with the target of achieving a medium stiffness range equivalent to that of a #3 PDE strut. Each strut was loaded to a predetermined safe deflection before yielding. Test results are shown in Tables 5 and 6.

TABLE 5
Test results for simplified Nylon 12 strut

| Force (lbf) | Position Y (in) | Position X | Δ Deflection Y | Δ Deflection X |
|---|---|---|---|---|
| 0 | 0.875 | 1.625 | 0 | 0 |
| 10 | 0.875 | 2.125 | 0 | 0.5 |
| 20 | 0.75 | 2.75 | −0.125 | 0.625 |
| 30 | 0.75 | 3.5 | 0 | 0.75 |
| 40 | 0.75 | 4.25 | 0 | 0.75 |
| Total | | | −0.125 | 2.625 |

The average stiffness in the X direction was 15.2 lbf/inch (1.71 Nm).

TABLE 6
Test results for optimized Nylon 12 strut

| Force (lbf) | Position Y (in) | Position X | Δ Deflection Y | Δ Deflection X |
|---|---|---|---|---|
| 0 | 2 | 1.25 | 0 | 0 |
| 10 | 2 | 1.5 | 0 | 0.25 |
| 20 | 2 | 2 | 0 | 0.5 |
| 30 | 2 | 2.25 | 0 | 0.25 |
| 40 | 2 | 2.625 | 0 | 0.375 |
| 50 | 2 | 3 | 0 | 0.375 |
| Total | | | 0 | 1.75 |

The average stiffness in the X direction was 28.6 lbf/inch (3.23 Nm).

Comparative measurements and imaging documented that the optimized strut achieved a medium stiffness range equivalent to the #3 PDE strut, thereby demonstrating that standard ankle-foot orthotic stiffness is attainable through the workflow outlined in FIG. 17.

All references, patents or applications, U.S. or foreign, cited in the application are, as a result of this, incorporated by reference as if written herein in their entirety. Where any inconsistencies arise, the material disclosed herein controls.

From the preceding description, one skilled in the art can easily ascertain the essential characteristics of this invention, and without departing from the spirit and scope thereof, can make various changes and modifications of the invention to adapt it to various usages and conditions.

What is claimed is:

1. A load-adaptive strut for an orthotic or prosthetic device, comprising:
a structural element configured by computational design using finite element analysis to adjust load transfer and energy return profiles based on simulation data derived from patient-specific parameters; and
adjustable connection points allowing variations in compound angles, offsets, and/or depths.

2. The strut of claim 1, wherein the structural element comprises a functionally graded lattice structure that exhibits controlled displacement tailored to the patient-specific parameters.

3. The strut of claim 1, wherein the adjustable connection points are configured for real-time automatic adjustment to update associated physics parameters.

4. The strut of claim 1, wherein the structural element is formed from one or more materials chosen from carbon fiber, advanced polymers, or composites thereof.

5. A load-adaptive strut for an orthotic or prosthetic device, comprising:
a means for adjusting load transfer and energy return profiles based on simulation data derived from patient-specific parameters; and
a means for adjusting connection points.

6. The strut of claim 5, wherein the means for adjusting load transfer and energy return profiles comprises a lattice structure that exhibits controlled displacement tailored to patient-specific parameters.

7. The strut of claim 5, wherein the means for adjusting connection points is operable to automatically update associated physics parameters in real-time.

8. The strut of claim 5, wherein the means for adjusting load transfer and energy return profiles comprises one or more materials chosen from carbon fiber, advanced polymers, and composites thereof.

9. A device for supporting or replacing a patient's limb, the device comprising:
the strut of claim 1; and
a structural assembly configured to modulate load distribution and energy return for the patient's limb.

10. The device of claim 9, wherein the structural assembly comprises an upper section and a lower section operatively coupled to the strut.

11. The device of claim 9, wherein the device is chosen from an orthotic device, a prosthetic device, an ankle-foot orthosis, a foot orthosis, a knee orthosis, and a knee-ankle-foot orthosis.

12. The device of claim 9, further comprising a configuration module for adjusting patient-specific design parameters.

13. The device of claim 12, wherein the patient-specific parameters include at least one input chosen from height, weight, activity level, center of mass, or gait parameters.

14. The device of claim 9, wherein the device is configured as a unibody orthosis formed as a single printed piece.

15. The device of claim 9, wherein the device is configured as an ankle-foot orthosis.

16. The device of claim 15, wherein the ankle-foot orthosis redistributes ground reaction forces to alleviate pain and enhance stability for weakened dorsiflexors and plantar flexors.

17. A method of designing a strut or device for supporting or replacing a patient's limb, the method comprising:
obtaining patient-specific parameters that include at least one value chosen from height, weight, activity level, center of mass, or gait parameters;
performing finite element analysis to determine a computationally designed geometry that modulates load transfer and energy return profiles; and
configuring adjustable connection points with compound angles, offsets, or depths based on simulation data.

18. The method of claim 17, further comprising automatically adapting the adjustable connection points in real-time to update physics parameters.

19. The method of claim 17, wherein the computationally designed geometry is applied to configure the device as an orthotic or prosthetic device.

20. A device formed by the method of claim 17.
